(19) United States
(12) Patent Application Publication
(10) Pub. No.: US 2026/0317784 A1
(43) Pub. Date: Oct. 15, 2026

(54) METHODS AND SYSTEMS FOR SYNTHETIC VIRUS PRODUCTION

(71) Applicant: THE UNIVERSITY OF BRITISH COLUMBIA, Vancouver (CA)

(72) Inventors: Nozomu YACHIE, Vancouver (CA); Samuel KING, Vancouver (CA); Soh ISHIGURO, Vancouver (CA); David MACKAY, Vancouver (CA)

(21) Appl. No.: 19/483,737

(22) PCT Filed: May 10, 2024
(86) PCT No.: PCT/CA2024/050636

§ 371 Date: Nov. 12, 2025

Related U.S. Application Data
(60) Provisional application No. 63/465,966, filed on May 12, 2023.

(51) Int. Cl. C12N 15/86

(52) U.S. Cl. CPC C12N 15/86; C12N 2740/15043; C12N 2740/15052; C12N 2760/20244

(57) ABSTRACT

A method and system for producing synthetic viruses are disclosed. The methods and systems of the present invention implement a plasmid recombination assay to reassemble split viral vectors to simultaneously reassemble the genome of a reassembled virus particle by a recombinase, and activate variant protein expression upon integration of a payload plasmid into a landing pad cell line. The landing pad cell line may be produced by integrating a landing pad plasmid into a genomic locus of a host cell line.

### CROSS-REFERENCE TO RELATED APPLICATIONS

This application claims priority from U.S. application No. 63/465,966 filed 12 May 2023 and entitled MULTIPLEXABLE PSEUDOVIRUS PRODUCTION FOR HIGH-THROUGHPUT VIRAL VARIANT SCREENING which is hereby incorporated herein by reference for all purposes. For purposes of the United States of America, this application claims the benefit under 35 U.S.C. § 119 of U.S. application No. 63/465,966 filed 12 May 2023 and entitled MULTIPLEXABLE PSEUDOVIRUS PRODUCTION FOR HIGH-THROUGHPUT VIRAL VARIANT SCREENING which is hereby incorporated herein by reference for all purposes.

### FIELD

This invention pertains to systems and methods of synthetic virus production, in particular, those that are produced from the reassembly of split viral vectors.

### BACKGROUND

Viruses are evolutionarily streamlined genetic units containing the minimal components necessary to survive. To reproduce, viruses hijack intracellular machinery, often killing or mutating the cells in their path. They are responsible for millions of deaths each year and cause at least a third of known infectious disease mortalities. To infect their hosts, viruses have evolved tropisms for different cell types, which are majorly dependent on the surface glycoproteins embedded in their envelope. Viral tropism is regarded as one of the most important factors for emerging and re-emerging viral diseases. For example, SARS-COV-2, the virus responsible for the global COVID-19 pandemic, presents a glycoprotein called the spike(S) protein on its surface, which interacts with the human ACE2 receptor on host cells to activate a fusion cascade leading to endocytosis of the viral particle. Given its essential function in the SARS-COV-2 life cycle, the S protein has evolved many of the key mutations leading to new, more infectious variants, such as the Omicron variant, which contains 15 unique amino acid changes in the S protein receptor binding domain. Therefore, studying variation in surface glycoproteins of existing and future viruses is crucial for effective medical intervention and understanding viral evolution.

Viral variation has been studied using protein interaction methods such as affinity purification and protein complementation, but these techniques are largely limited to analyzing individual proteins at a time, may suffer from off-target effects, require synthetic protein fusions, or function in artificial contexts far from a real viral infection environment. Surface display systems such as yeast display and phage display have proven useful for scaling the number of proteins analyzed in parallel; however, they are prone to non-native protein modifications or cannot facilitate direct development of viral vectors. For these reasons, viral pseudotyping—the process of generating synthetic viruses with variant glycoproteins on their surface—has become a popular method of choice for researching viral variation and has greatly expanded our capacity to alter and study cellular and molecular systems.

Retroviruses, a class of viruses that can integrate their own genome into the genome of host cells they have infected, has allowed for safe design and generation of pseudotyped synthetic viruses in vitro.

The term “pseudotyping” or “pseudotyped virus” refers to an enveloped virus that has incorporated into its envelope a heterologous envelope (env) glycoprotein.

Retroviruses can be engineered as vectors to introduce genetic parts to a cell, which is useful for delivering therapeutic agents or for modifying a host cell's genome. One example of a retroviral vector is the lentiviral vector (LV) system developed from human immunodeficiency virus. To produce pseudotyped LVs, the LV genome, packaging proteins, and a chosen surface glycoprotein is exogenously expressed in the cell, which accumulates in the plasma membrane and decorates budding virions as they depart from the cell. Synthetic viruses in the cell culture media are then harvested for targeting of broad or specific cell types. For broad infectivity, the vesicular stomatitis virus G (VSV-G) glycoprotein, which has tropism for the human low-density lipoprotein receptor (LDL-R), may be used for LV engineering in mammalian systems due to the nearly ubiquitous expression of LDL-R in many cell types. VSV-G can be commonly expressed when generating any batch of LVs; however, for targeting specific cell types, each variant protein must be expressed only with their intended LV genome to package a monoclonal virus. Individual packaging and testing of each pseudotyped variant may be feasible at the scale of approximately hundreds of variants, but it is a laborious and resource-intensive process that limits our ability to study observed and unobserved viral variants at the scale necessary for medicinal development and understanding viral evolution (FIG. 1A). If pseudotyping plasmids were pooled with transfer and packaging plasmids in the same cells, polyclonal viruses decorated with multiple pseudotypes would be produced (FIG. 1B), eliminating the ability to use the LVs for variant analyses.

DNA barcoding has become a staple of multiplexed biology, greatly increasing the throughput of technologies ranging from next-generation sequencing to perturbation screening. Barcoding the LV genome allows for the combination of LVs to be interpreted in parallel by a one-shot deep amplicon sequencing of the barcodes integrated in host cell genomes. While pooling barcoded LVs permits analysis of many variants' infectivity at once, it still does not allow for multiplexable production of barcoded variants. A recent system was proposed for maintaining surface variant-barcode associations during viral packaging but relies on individual packaging and co-transduction of known variant-barcode combinations and cannot efficiently accommodate large variant sizes due to decreases in LV packaging rate.

The inventors have recognized a general need for an improved synthetic virus production system. There is a particular need for an improved synthetic virus production system which allows for multiplexable production of pseudotyped viruses.

### SUMMARY

Aspects of the invention relate to methods and systems for producing synthetic viruses. The methods and systems of the present invention implements a plasmid recombination assay to reassemble split viral vectors to simultaneously reassemble the genome of a reassembled virus particle by a recombinase, and activate variant protein expression upon integration of a payload plasmid into a landing pad cell line.

The landing pad plasmid encodes a first segment of a genome of a virus particle. The payload plasmid encodes a second segment of the genome of the virus particle. The recombining of the nucleic acid sequences contained in the landing pad plasmid and the payload plasmid produces a reassembled virus particle comprising the viral genome formed by the first and second segments.

The payload plasmid encodes a variant protein. In some embodiments, the variant protein is a surface glycoprotein. In some embodiments, the payload plasmid encodes a unique barcode sequence. In some embodiments, the unique barcode sequence is encoded within the genome of the reassembled virus particle. Each unique barcode sequence may correspond to one variant protein.

In some embodiments, the variant protein is encoded outside of the genome of the reassembled virus particle.

In some embodiments, a functional virus is produced from transfecting a packaging plasmid into the payload- and landing pad-integrated cell. Each functional virus may comprise one unique barcode sequence encoded within the viral genome. Each functional virus may become active with the incorporation of one variant protein and used for infection into a target cell.

The virus particle is in some embodiments a lentivirus.

One example application of the systems and methods of the present invention is in the production of a library of pseudotyped viruses. Aspects of the present invention provides a multiplexable, barcoded synthetic virus packaging method and system, which enables large scale and high-throughput screening of a library of viral variants. The ability to perform a large-scale screen of infectious variants is beneficial in understanding and providing the ability to predict the evolution of viruses. This may assist with preventive pandemic measures such as for vaccine design. One non-limiting example application of the present invention is in the design of vaccines which target the new variants against the S protein of the coronavirus.

Further aspects and example embodiments are illustrated in the accompanying drawings and/or described in the following description.

It is emphasized that the invention relates to all combinations of the above features, even if these are recited in different claims.

### BRIEF DESCRIPTION OF THE DRAWINGS

The accompanying drawings illustrate non-limiting example embodiments of the invention.

FIG. 1A is a schematic diagram illustrating a prior art process for one-by-one packaging of viral variants. The FIG. 1A prior art process has low throughput but retains monoclonal identities of viruses.

FIG. 1B is a schematic diagram illustrating a prior art process for pooled packaging of viral variants. The FIG. 1B process has high throughput but the identities of viruses are polyclonal and cannot be discerned from each other.

FIG. 1C is a schematic diagram illustrating a multiplexable process for producing pseudotyped viruses according to an example embodiment of the invention. This process or genetic circuit may be referred to herein as the “GHOST: a genetic circuit for highly scalable pseudovirus production”. The multiplexable process of the present invention utilizes an engineered cell line, which advantageously enables high throughput production of monoclonal viruses.

FIG. 1D is a schematic diagram illustrating a payload plasmid, a landing pad plasmid, and the recombined sequence comprising the reassembled viral genome and activation of the variant gene expression, according to an example embodiment of the invention. BC=barcode; NGS=next-generation sequencing; Var.=variant; vBC=variant barcode.

FIG. 1E is a schematic diagram illustrating a pseudovirus with a fusogen of interest mediated by GHOST and a corresponding receptor of interest, which is expected to emulate viral infection at scale.

FIG. 2A is a schematic diagram illustrating a payload plasmid, a landing pad plasmid, and the recombined sequence comprising the reassembled viral genome and activation of the variant gene expression, according to an example embodiment of the invention. FIG. 2A illustrates schematically a plasmid recombination assay to reassemble split lentiviral vectors according to an example embodiment of the invention.

FIG. 2B is a payload plasmid used in the Examples, which the results are shown in FIGS. 2C to 2E.

FIG. 2C are representative Brightfield and fluorescence microscopy images of Lenti-X HEK293T cells three days after transfection of the FIG. 2B payload plasmid into the landing pad cell line.

FIG. 2D are plots of mean fluorescence intensity (MFI) for StayGold and mTagBFP2 (n=3).

FIG. 2E are plots of percent fluorescent protein (FP)-positive cells three days after transfection (n=3).

FIG. 2F is a payload plasmid used in the Examples, which the results are shown in FIG. 2G, 3D to 3F, and 3N to 3O.

FIG. 2G are plots which show transduction efficiencies of reassembled viral vectors based on StayGold fluorescence after infection (n=3). VSV-G supplemented conditions were transfected with both VSV-G and packaging plasmid after payload-landing pad recombination. VSV-G on payload conditions had VSV-G encoded on the payload plasmid and were transfected with only a packaging plasmid after payload-landing pad recombination. Statistics=one-way ANOVA with Tukey HSD and Bonferroni correction for 6 (VSV-G supplemented) or 5 (VSV-G on payload) groups.

FIG. 2H are representative fluorescence microscopy images of Lenti-X HEK293T cells three days after transfection of Bxb1 or Pa01 payload and landing pad plasmids

FIG. 2I show the fold difference in StayGold MFI of Pa01 versus Bxb1 payload integrations across four payload sizes. All scale bars, 50 μm. All error bars represent mean±95% confidence interval.

FIG. 3A is a schematic diagram illustrating a method of generating the landing pad cell line according to an example embodiment. The FIG. 3A method illustrates TALEN knock-in and homology-directed repair of the landing pad plasmid in the AAVS1 safe harbor locus of host cell genomes. Landing pad genotyping primers are shown as small arrows (i.e., a, b, c, d, b′, c′). HA-L=left homology arm; HA-R=right homology arm.

FIG. 3B are representative Brightfield (BF) and fluorescence microscopy images of landing pad cells at day 0 and day 13 of Blasticidin selection, with a magnified image of a single resistant colony. Scale bars, 200 μm. Magnified scale bars, 50 μm.

FIG. 3C illustrates an example timeline of landing pad cell line generation.

FIG. 3D are representative Brightfield (BF) and fluorescence microscopy images of payload-integrated wildtype Lenti-X HEK293T (WT) and Blasticidin-selected landing pad (LP) cells at Day 21 after transfection of payload. Scale bars, 50 μm.

FIG. 3E are plots depicting Staygold intensity of three replicates pooled of wildtype Lenti-X HEK293T (WT) or Blasticidin-selected landing pad (LP) cells from FIG. 3D at Day 29 after transfection with the payload plasmid.

FIG. 3F are plots showing the percent StayGold positive cells three days after transduction with virus produced using cells from FIG. 3E transfected with varying amounts of packaging (psPAX2), VSV-G (pMD2.G), or proviral (pBC008) plasmid. 2* indicates virus was pooled from 3 wells each transfected with 2 μg packaging plasmid. Exp.=experiment.

FIG. 3G is a schematic diagram illustrating an example landing pad clone isolation procedure.

FIG. 3H is a chart illustrating a summary of clones isolated from the unsorted Blasticidin-selected landing pad cells.

FIG. 3I illustrate mCherry intensities of all landing pad clones from two independent 96-well single-cell sorts using Blasticidin-selected cells from the results of FIG. 3B. BS=bulk sorted landing pad cells; C=clone; UN=unsorted Blasticidin-selected landing pad cells; WT=wildtype Lenti-X HEK293T cells.

FIG. 3J depicts the results of genotyping analysis of β-actin, the LP plasmid, and the landing pad-integrated and wildtype AAVS1 locus. Primer locations are shown in FIG. 3A. bp=base pairs. LP=landing pad plasmid. UN=unsorted Blasticidin-selected landing pad cells; WT=wildtype Lenti-X HEK293T cells.

FIG. 3K is a schematic diagram illustrating a TALEN-induced double-strand break and non-homologous end joining (NHEJ) at the AAVS1 locus.

FIG. 3L illustrates 40 bp Sanger sequencing results of the left and right homology arms using primers a and d from UN in FIG. 3J.

FIG. 3M illustrates 20 bp Sanger sequencing results of the left and right homology arms using primers a and d from C24 and 35 in FIG. 3J.

FIG. 3N are plots depicting Staygold intensity of three replicates (rep.) of wildtype Lenti-X HEK293T (WT), unsorted Blasticidin-selected landing pad (UN), candidate C24 and C35 cells at Day 22 after transfection with the payload plasmid.

FIG. 3O is a plot showing the percent StayGold positive cells three days after transduction with virus produced using replicate 2, 1, and 3 of wildtype Lenti-X HEK293T (WT), unsorted Blasticidin-selected landing pad (UN), and candidate C35 cells, respectively, from FIG. 3N transfected with packaging (psPAX2) plasmid. Virus was pooled from 3 wells each transfected with 2 μg packaging plasmid.

FIG. 4A is a schematic diagram illustrating a method of generating the landing pad cell line according to another example embodiment. The FIG. 4A method illustrates a lentivirus (LV) integration strategy. IpBC=landing pad barcode; MOI=multiplicity of infection.

FIG. 4B is a schematic diagram illustrating plasmid recombination of a payload plasmid comprising a split Puromycin and StayGold reporter with the landing pad plasmid containing the complementary half of Puromycin according to an example embodiment of the invention.

FIG. 5 is a schematic diagram illustrating downstream assays which may be performed on the library of functional viruses produced using the methods and systems according to example embodiments of the invention.

FIG. 6A is a schematic diagram illustrating plasmid recombination of a payload plasmid comprising a StayGold reporter with a landing pad plasmid according to an example embodiment of the invention.

FIG. 6B are representative Brightfield and fluorescence microscopy images of HEK293TN cells after transfection of zsGreen, EGFP, or StayGold.

FIG. 6C is a plot of mean fluorescence intensity (MFI) of cells after transfection of zsGreen, EGFP, or StayGold.

FIG. 6D are representative Brightfield and fluorescence microscopy images of Lenti-X HEK293T cells three days after transfection of a combination of Bxb1 integrase, landing pad, and payload plasmids.

FIG. 6E is a plot of StayGold MFI of the cells described in FIG. 6D (n=2).

FIG. 6F is a plot of StayGold MFI three days after transfection when Bxb1 integrase is encoded on the landing pad (LP). All scale bars, 200 μm. All error bars represent mean±95% confidence interval.

FIG. 7A are representative Brightfield and fluorescence microscopy images of HEK293TN cells two days after transduction with VSV-G-pseudotyped third-generation lentivirus (RSV promoter) or second-generation lentivirus (Tat promoter).

FIG. 7B is a plot of % EGFP positive cells as a function of relative viral dilutions to depict transduction efficiencies of third-generation and second-generation lentiviruses across 0-60 μL of virus, measured by percent GFP-positive cells (n=2).

FIG. 7C are representative Brightfield and fluorescence microscopy images of HEK293Ta cells four days after transduction with VSV-G-pseudotyped second-generation lentivirus produced from either HEK293Ta cells or Lenti-X HEK293T cells.

FIG. 7D are representative Brightfield and fluorescence microscopy images of HEK293TN cells two days after transduction with VSV-G pseudotyped second-generation lentivirus produced from either HEK293TN cells or Lenti-X HEK293T cells.

FIG. 7E are plots of % green-positive cells as a function of relative viral dilution, illustrating viral titer of second-generation lentiviruses produced from HEK293TN or Lenti-X HEK293T cells (n=2). Virus 1, 5.5 kb; virus 2, 6.7 kb. All scale bars, 200 μm. All error bars represent mean±95% confidence interval.

FIG. 8A are representative flow cytometry gating of transduced cells from FIG. 2G.

FIG. 8B are representative flow cytometry gating of transduced cells from Exp. 1 in FIG. 3F.

FIG. 9 is a flow chart illustrating a method of producing a synthetic virus according to an example embodiment of the invention.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

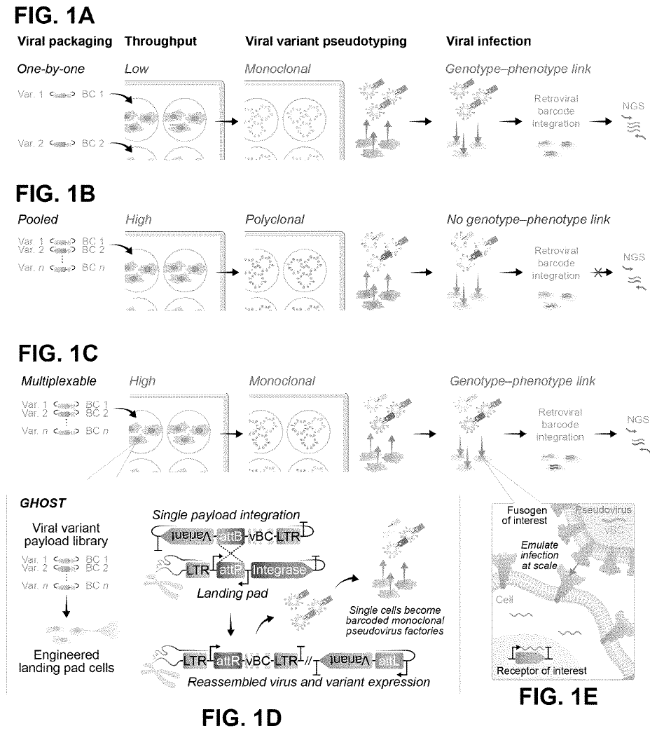

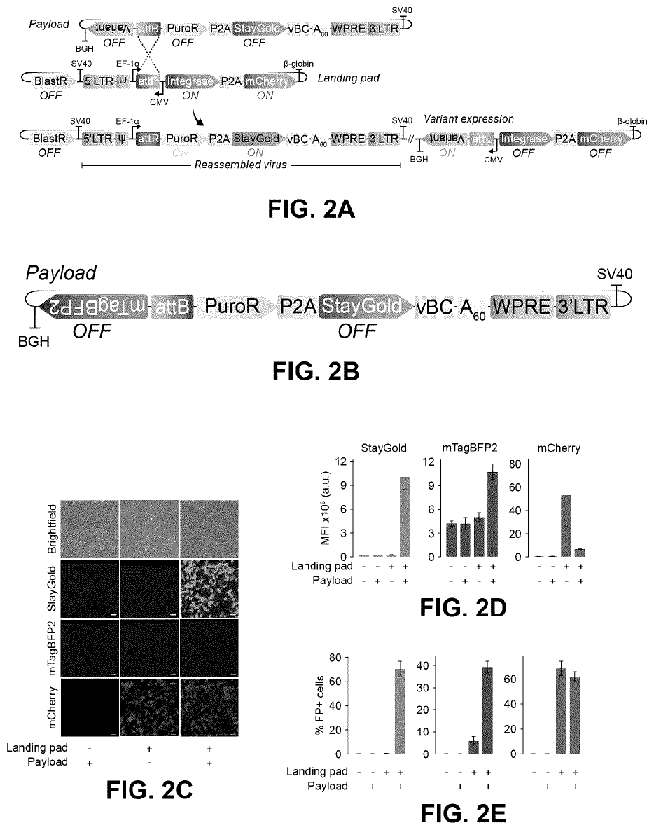

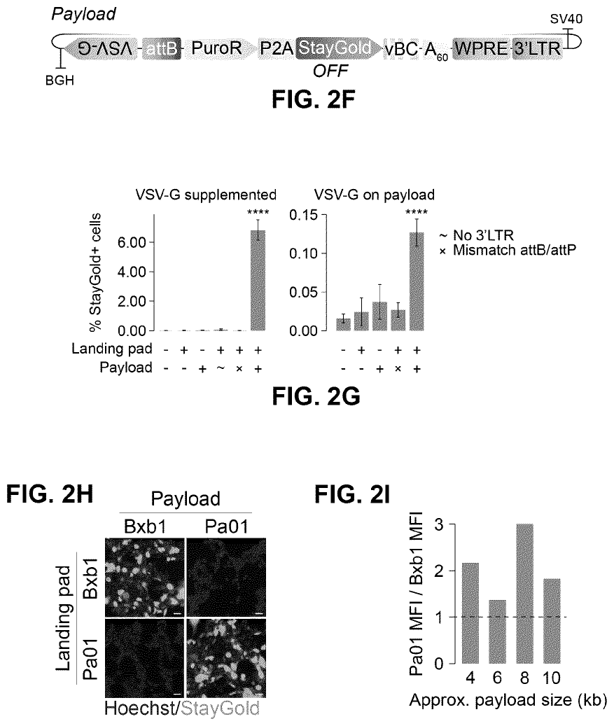

Aspects of the invention relate to systems for producing synthetic viruses. FIGS. 1D and 2A illustrates example systems of producing synthetic viruses. The system comprises a landing pad cell line and a payload plasmid.

The landing pad cell may be engineered from a host cell line. In some embodiments, a landing pad plasmid is integrated into a genomic locus of the host cell line. The landing pad plasmid may be transfected into the host cell line. In some embodiments, the host cell line is a eukaryotic cell, and in some embodiments, a mammalian cell. Non-limiting examples of cell types that may be used as the host cell include HEK 293 cells, HEK 293T cells, HEK293TN cells, HEK293FT cells, COS cells, NSO cells, etc.

FIGS. 1D and 2A illustrate landing pad plasmids according to some example embodiments of the invention. In some embodiments, the landing pad plasmid comprises a nucleic acid sequence having a first segment of a genome of a virus particle, and a first recombination site.

In some embodiments, the first segment of the genome of the virus particle comprises a sequence of a long terminal repeat (LTR) of the virus particle. In some embodiments, the virus particle is a retrovirus, such as a lentivirus. In such embodiments, the term “long terminal repeat” or “LTR” refers to domains of base pairs located at both 5′ and 3′ ends of retroviral DNAs, which are direct repeats. The viral LTR comprises U3, R, and U5 regions which are involved in transcription of a viral gene, reverse transcription from a viral genome, and integration of a double-stranded DNA synthesized via the reverse transcription into a host DNA.

In some embodiments, the first segment of the genome of the virus particle comprises the nucleic acid sequence of the 5′LTR of the virus particle. In some embodiments, the first segment of the genome of the virus particle comprises the nucleic acid sequences of the 5′LTR of the virus particle, and additionally one or more of a Psi packaging element (Psi Y), Rev responsive element (RRE), Tat responsive element (TAR), and Central polypurine tract (cPPT).

A “recombination site” refers to a recognition sequence on a nucleic acid molecule that participates in an integration/recombination reaction by recombination proteins such as a recombinase. Non-limiting examples of recombination sites include attB sites, attP sites, attL sites, attR sites, lox sites, psi sites, dif sites, cer sites, frt sites, and portions, mutants, variants, and derivatives thereof.

In some example embodiments, the first recombination site comprises an attP site, or portions, mutants, variants, and derivatives thereof.

In some embodiments, a promoter is operably linked to the first recombination site.

In some embodiments, the promoter is a constitutive promoter. Non-limiting examples of suitable constitutive promoters include a promoter selected from the group consisting of an EF-1alpha promoter, a PGK-1 promoter, a UBC promoter, a CMV promoter, a CAGG promoter and an SV40 promoter.

In some embodiments, the promoter is an inducible promoter. Non-limiting examples of suitable inducible promoters include a NFAT promoter, a STAT3-sensing promoter, a CD69 promoter, a CD137 promoter, a hypoxia responsive element promoter, and a minimal promoter operably linked to an inducible enhancer.

In some example embodiments, an EF-1alpha promoter is operably linked to the first recombination site.

In some embodiments, two promoters are operably linked to the first recombination site. The first promoter may be operably linked upstream of the first recombination site in a first direction. The second promoter may be operably linked downstream of the first recombination site in an opposing second direction. The first and second promoters may be unidirectional. The first and second promoters may be a constitutive and/or an inducible promoter. The first and second promoters may be the same promoter or different promoters. In some example embodiments, the first and second promoters are EF-1alpha promoter and a CMV promoter respectively.

In some embodiments, the landing pad plasmid further comprises a nucleic acid sequence which encodes a recombinase. The recombinase may comprise one or more site-specific recombinases. The recombinase may be any suitable recombinases which recognize and bind two recombination sites and mediate recombination between these two target sites. Non-limiting examples of recombinases include one or more serine recombinases and/or tyrosine recombinases, such as but not limited to Bxb1, Pa01, Hin, Gin, Tn3, β-six, CinH, ParA, γδ, φC31, TP901, TG1, φBT1, R4, φRV1, φFC1, MR11, A118, U153, gp29, Cre, FLP, R, Lambda, HK101, HK022, and pSAM2, etc. In some embodiments, the recombinase comprises one or more members of the large serine recombinase family such as BxB1, BxZ2, PhiC31, Peaches, Veracruz, Rebeuca, Theia, Benedict, PattyP, Trouble, KSSJEB, Lockley, Scowl, Switzer, Bob3, Abrogate, Doom, ConceptII, Anglerfish, SkiPole, Museum, and Severus, etc

In some embodiments, the first recombination site is positioned between the nucleic acid sequence of the LTR and the sequence which encodes the recombinase. As used herein, when it is referred to a sequence being positioned “between” two sequences, the sequence may be, but need not be, positioned immediately adjacent to (or flanking) the two sequences.

In some embodiments, the landing pad plasmid is integrated into a safe harbor locus of the host cells. The safe harbor locus may for example be AAVS1, CCR5, ROSA26, collagen, HTRP, H11, beta-2 microglobulin, GAPDH, TCR, RUNX1, and/or ALB.

In some embodiments, the landing pad plasmid is integrated into a transcriptional unit of the host cell. The transcriptional unit refers to a DNA segment that is transcribed into a RNA from a promoter.

FIGS. 1D and 2A illustrate payload plasmids according to some example embodiments of the invention. In some embodiments, the payload plasmid comprises a nucleic acid sequence having a second segment of a genome of a virus particle, a second recombination site, and a nucleic acid sequence which encodes a variant protein.

In some embodiments, the second segment of the genome of the virus particle comprises a sequence of a long terminal repeat (LTR) of the virus particle. In some embodiments, the second segment of the genome of the virus particle comprises the nucleic acid sequence of the 3′LTR of the virus particle. In some embodiments, the second segment of the genome of the virus particle comprises the nucleic acid sequences of the 3′LTR of the virus particle, and a posttranscriptional regulatory element sequence. In some example embodiments, the posttranscriptional regulatory element sequence comprises a Woodchuck Hepatitis Virus (WHP) Posttranscriptional Regulatory Element (WPRE) sequence. However, it is understood that other suitable transcriptional regulatory element sequences may also be used, for example, posttranscriptional regulatory element sequences of Hepatitis B virus (HPRE).

The second recombination site on the payload plasmid pairs with the first recombination site on the landing pad plasmid. In some example embodiments, the first recombination site comprises an attP site, or portions, mutants, variants, and derivatives thereof, and the second recombination site comprises an attB site, or portions, mutants, variants, and derivatives thereof. In some embodiments, the landing pad plasmid comprises a nucleic acid sequence which encodes a recombinase which is specific for the paired first and second recombination sites.

The nucleic acid sequence which encodes the variant protein may be any gene or protein of interest. In some embodiments, the variant protein comprises a viral envelope protein. In some embodiments, the variant protein comprises an envelope glycoprotein. In some embodiments, a library of payload plasmids each comprising a predetermined variant nucleic acid which encodes a variant protein is formed.

In some embodiments, the payload plasmid further comprises a unique nucleic acid barcode identifier, or unique barcode sequence. In such embodiments, each of the payload plasmids in a library of payload plasmids comprises a unique barcode sequence, or a differentiating barcode sequence. In some embodiments, the unique barcode sequence comprises about 20 to about 100 basepairs in length, and in some embodiments, about 40 to 60 basepairs in length, and in some embodiments, about 8 to 16 basepairs in length. In some embodiments, the unique barcode sequence comprises a DNA sequence with the formula, (SW)ₙ, wherein S is guanine (G) or cytosine (C), and W is adenine (A) or thymine (T), and n is at least 1. In some embodiments, n is 10 to 50, and in some embodiments, 15 to 40, and in some embodiments, 20 to 30. In some embodiments, the unique barcode sequence comprises a DNA sequence with the formula, (N)ₙ, wherein N is A, G, T, or C, and n is at least 1. In some embodiments, n is 10 to 50, and in some embodiments, 15 to 40, and in some embodiments, 20 to 30.

In some embodiments, the second recombination site is between the nucleic acid which encodes the variant protein and the unique barcode sequence. In some embodiments, the unique barcode sequence is between the LTR and the second recombination site.

In some embodiments, the landing pad plasmid and/or the payload plasmid further comprise nucleic acid sequences which encode one or more selectable markers and/or reporter proteins. The selectable markers and/or reporter proteins facilitate the sorting, after the transfecting of the payload plasmid into the landing cell line, of transfected cells which comprise the integrated plasmids, from untransfected cells, or generally cells which do not comprise the integrated plasmids. In some embodiments, the one or more selectable markers comprise an antibiotic resistance marker. The antibiotic resistance marker may confer resistance to an antibiotic, such as G418, hygromycin, blasticidin, neomycin, puromycin, etc. In some embodiments, the one or more reporter proteins comprise a fluorescent reporter protein. The fluorescent reporter protein generates a fluorescence signal upon being excited to a light energy at a wavelength.

In some embodiments, the system for producing a library of viral vectors further comprises a packaging plasmid. The landing pad plasmid, the payload plasmid, and the packaging plasmid may be integrated to form functional viral vectors.

The packaging plasmid comprises nucleic acid sequences which encode viral structural proteins. In some embodiments, the packaging plasmid comprises nucleic acid sequences which encode Gag and/or Pol, or variants, derivatives and mutations thereof. The packaging plasmid may additionally comprise nucleic acid sequences which encode one or more regulatory/accessory proteins such as a trans-activator of transcription (Tat) protein, a Rev protein, and a Rev response element (RRE), or variants, derivatives and mutations thereof. The packaging plasmid further comprises suitable one or more promoter(s) operably linked to control the expression of the proteins (e.g., Gag, Pol, Tat, Rev, RRE, etc.). The one or more promoters may be a constitutive promoter and/or an inducible promoter. One non-limiting example of packaging plasmid that may be suitable for use in the viral vector packaging system is psPAX2 (Addgene plasmid #12260), but other suitable packaging plasmids may be used, such as pCMV delta R8.2 (Addgene plasmid #12263) and pCMV-dR8.2 dvpr (Addgene plasmid #8455).

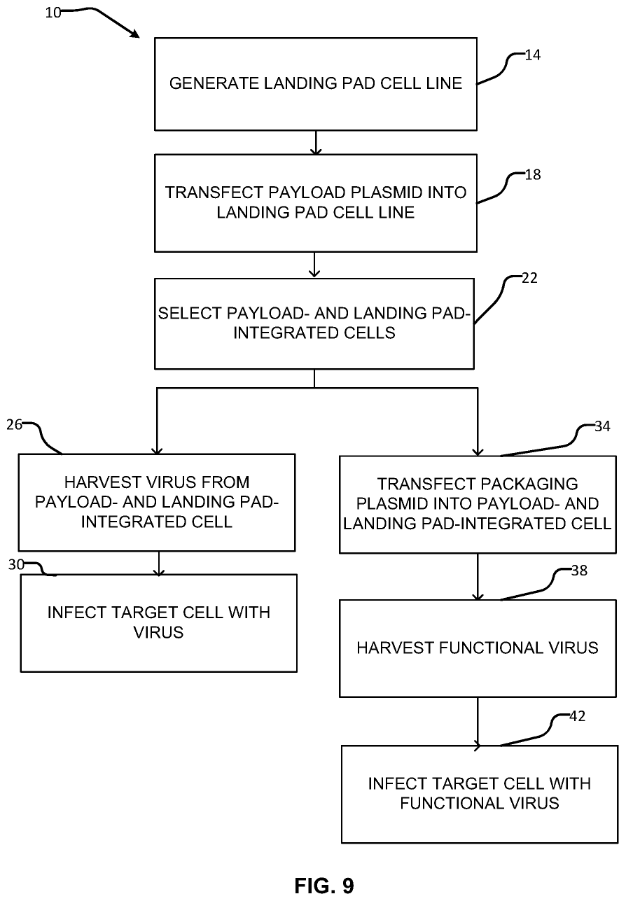

Aspects of the invention relate to methods of generating a synthetic virus. Referring to FIG. 9, in some embodiments, the method of generating a synthetic virus comprises generating a landing pad cell line (block 14). In some embodiments, the generating of the landing pad cell line comprises transfecting the landing pad plasmid into the host cell line. The transfecting of the landing pad plasmid into the host cell line comprises integrating the landing pad plasmid into a genomic locus of the host cell line.

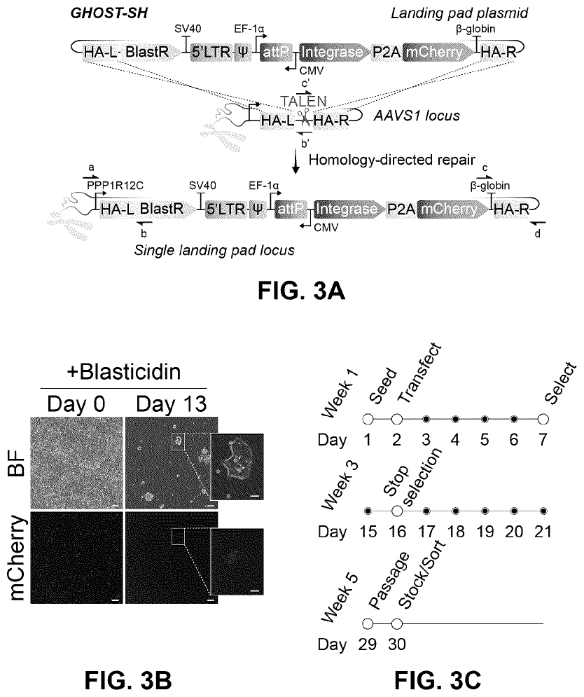

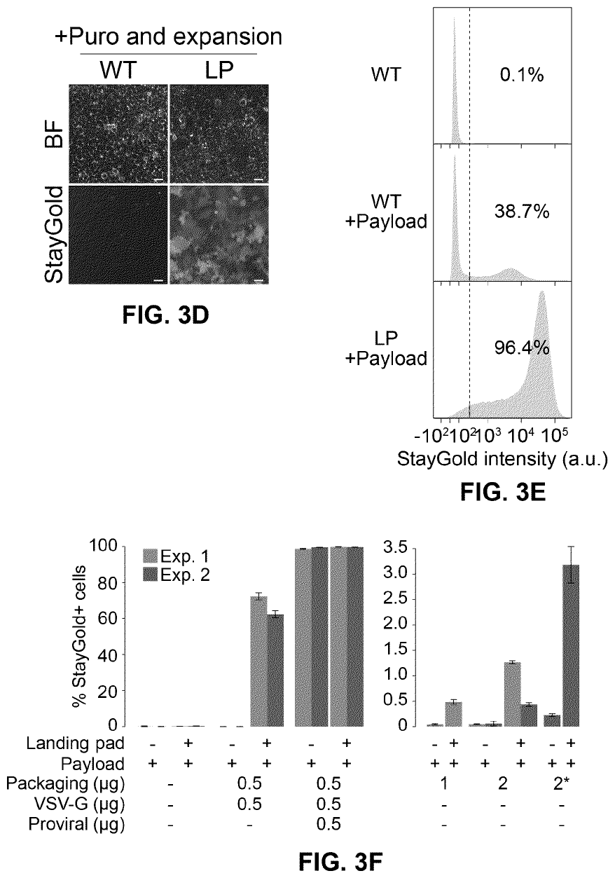

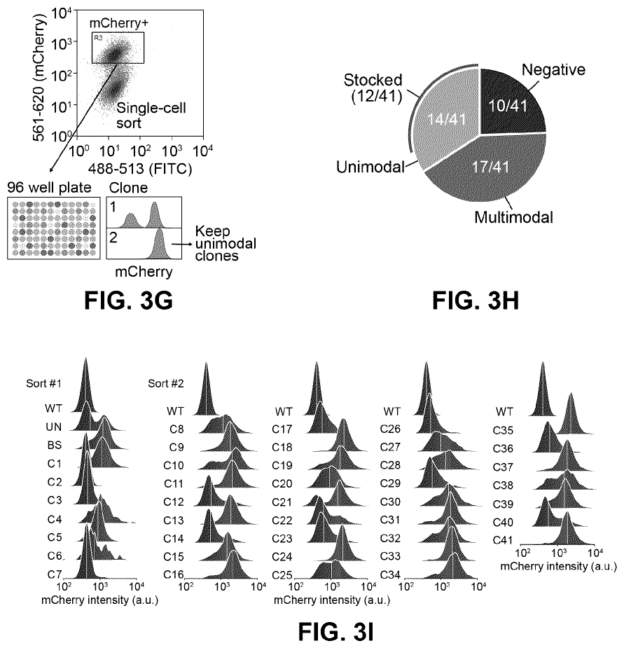

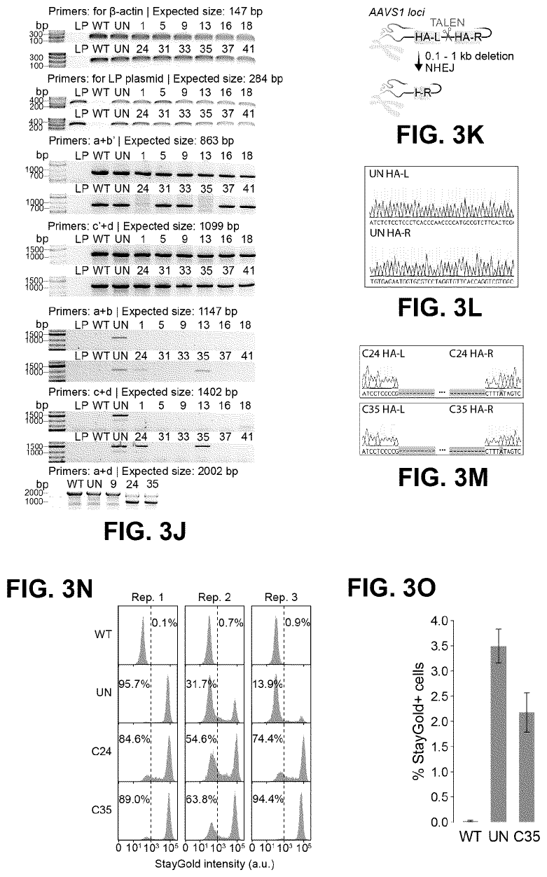

Referring to the schematic illustration in FIG. 3A, in some example embodiments, homology-directed repair (HDR) is used as a method to insert the landing pad plasmid into the genomic locus of the host cell line. In such embodiments, the landing pad plasmid comprises a repair template for homology-directed repair. In some embodiments, the nucleic acid sequences of the LTR, the first recombination site, the nucleic acid sequence which encodes the recombinase, and/or the reporter protein(s) and/or selectable markers may be flanked by homology arms. The cleavage or break of the nucleic acid sequence may be created by any suitable nucleases, such as a TAL-effector domain nuclease (TALEN). In some embodiments, the landing pad plasmid is integrated into a safe harbor locus of the host cell genome. In some example embodiments, the safe harbor locus is AAVS1. This method may be referred to herein as “GHOST-SH”.

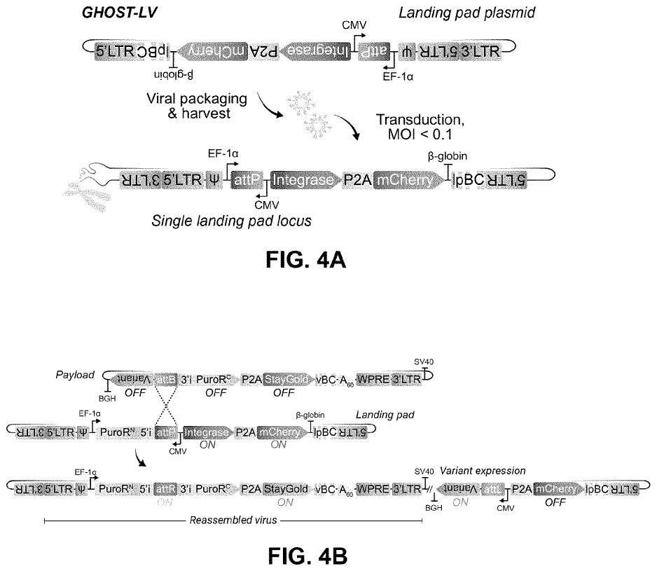

Referring to the schematic illustration in FIGS. 4A and 4B in some example embodiments, lentivirus transduction is used as a method to insert the landing pad plasmid into the genomic locus of the host cell line. In such embodiments, the landing pad plasmid comprises the 5′ LTR and 3′LTR of the lentivirus genome. In some embodiments, the nucleic acid sequences of the LTR, the first recombination site, the nucleic acid sequence which encodes the recombinase, and/or the reporter protein(s) and/or selectable marker(s) may be flanked by the 5′ LTR and 3′LTR of the lentivirus genome. In some embodiments, the landing pad plasmid additionally comprises a unique landing pad barcode sequence. In some embodiments, the unique landing pad barcode sequence is positioned adjacent to the sequence of the 5′LTR of the lentivirus genome. This method may be referred to herein as “GHOST-LV”.

Other suitable genome editing methods and systems may be used to integrate the landing pad plasmid into the genomic locus of the host cell line.

In block 18, a payload plasmid may be transfected into the landing pad cell line to produce a payload- and landing pad-integrated cell. The transfecting of the payload plasmid into the landing pad cell line comprises recombining the sequences contained in the landing pad plasmid and the payload plasmid in the presence of a recombinase. In some embodiments, the transfecting of the payload plasmid into the landing pad cell line comprises recombining the sequences contained in the landing pad plasmid and the payload plasmid at the first and second recombination sites to form first and second recombined sites, respectively. In some example embodiments, the first and second recombination sites are attP and attB, or derivatives thereof, and the first and second recombined sites are attL and attR, or derivatives thereof.

In some embodiments, the recombining of the sequences contained in the landing pad plasmid and the payload plasmid forms a reassembled vector comprising the provirus, or the genome of the reassembled virus particle. The genome of the reassembled virus particle may comprise the recombined first and second segments of the genome of the virus particle. In some embodiments, the unique barcode sequence is encoded in the genome of the reassembled virus particle.

In some embodiments, the recombining of the sequences contained in the landing pad plasmid and the payload plasmid forms a reassembled vector comprising a nucleic acid sequence encoding the variant protein. In some embodiments, the nucleic acid sequence which encodes the variant protein is not contained in the (i.e., outside of the) genome of the reassembled virus particle. In some embodiments, the nucleic acid sequence encoding the variant protein is positioned upstream or downstream of the genome of the reassembled virus particle. In some embodiments, the expression of the variant protein is activated upon recombination of the payload plasmid with the landing pad plasmid.

FIGS. 1D and 2A illustrate schematically a reassembled vector according to an example embodiment of the invention. In some example embodiments, the first and second recombination sites are attP and attB, respectively, and the first and second recombined sites are attL and attR, respectively.

In such example embodiments, the reassembled vector comprises the first segment of the genome of the virus particle comprising the nucleic acid sequence of the 5′LTR of the virus particle nucleic acid sequence, the second segment of the genome of the virus particle comprises the nucleic acid sequence of the 3′LTR of the virus particle, and therebetween, the second recombined site (e.g., attR) and the unique barcode sequence. In some embodiments, the first promoter is operably linked to the second recombined site. In some embodiments, the reassembled vector additionally comprises a nucleic acid sequence which encodes a selectable marker and a reporter protein. The nucleic acid sequences which encode the selectable marker and the reporter protein may be positioned between the second recombined site (e.g., attR) and the 3′LTR of the virus particle nucleic acid sequence.

In such example embodiments, the reassembled vector additionally comprises the nucleic acid which encodes the variant protein, the recombinase, and the first recombined site (e.g., attL). The nucleic acid which encodes the variant protein is in some embodiments outside of the genome of the virus particle. In some embodiments, the nucleic acid which encodes the variant protein is in some embodiments positioned downstream of the 3′LTR of the virus particle nucleic acid sequence, in a direction opposite to the 5′LTR of the virus particle nucleic acid sequence. In some embodiments, the second promoter is operably linked to the first recombined site. In some embodiments, the reassembled vector additionally comprises a nucleic acid sequence which encodes a reporter protein. The nucleic acid sequence which encodes the reporter protein may be positioned downstream of the nucleic acid sequences which encodes the variant protein, the recombinase, and/or the first recombined site (e.g., attL).

The payload- and landing pad-integrated cell may be selected using the one or more selectable markers and/or the one or more reporting proteins (block 22).

In some embodiments, the virus from the payload- and landing pad-integrated cell or the selected payload- and landing pad-integrated cell is harvested (block 26). The harvested virus may be introduced to a target cell line, e.g., by infecting the target cell line with the virus (block 30). The expressed variant proteins may be screened for at least one predetermined property, characteristic or activity. One non-limiting example of such predetermined property, characteristic or activity is assessing the infectivity of certain variant protein to the target cell line.

In some embodiments, a packaging plasmid is transfected into the payload- and landing pad-integrated cell or the selected payload- and landing pad-integrated cell to produce a transfected cell (block 34). A functional virus may be harvested from the transfected cell (block 38). The functional virus may be transduced into a target cell line (block 42).

The target cell line may be any eukaryotic cell line which may be desirable for use to screen the virus. In some embodiments, the target cell line is a mammalian cell line.

The systems and methods of the present invention may be used to produce a library of variant proteins.

The systems and methods of the present invention may be used for pseudotyping. In some aspects of the invention, the functional viruses comprise pseudotyped viruses. In some embodiments, a library of functional viruses may be harvested after the transfection of packaging plasmids into the payload- and landing pad-integrated cells formed by transfecting a library of payload plasmids into the landing pad cell line. The library of functional viruses each comprises a surface protein which is encoded by the nucleic acid encoding the variant protein. In some embodiments, the surface protein comprises a surface glycoprotein. In some example embodiments, the surface glycoprotein is a spike(S) protein.

Aspects of the present invention provides a multiplexable, barcoded synthetic virus packaging method and system, which enables large scale and high-throughput screening of a library of viral variants.

In some embodiments, the functional viruses are monoclonal viruses. A monoclonal virus contains one or more copies of one variant protein. In some embodiments, at least some of the functional viruses are monoclonal viruses, and in some embodiments, at least half, and in some embodiments, a majority. As used herein, “half” means 50 percent (%) of the total number of functional viruses in the library of functional viruses produced, and “majority” means 75% of the total number of functional viruses in the library of functional viruses produced.

In some embodiments, the transfecting of the landing pad plasmid into the host cell line in block 14 comprises integrating one landing pad plasmid into the genomic locus of the host cell line. The integration of one landing pad plasmid into the genomic locus of the host cell ensures that each transfected cell produces a monoclonal virus.

In some embodiments, the method further comprises verifying single landing pad integration into the host cell line. The suitable verification method may depend on the system that is used to transfect the landing pad plasmid into the host cell line. Any suitable verification methods may be used, including but not limited to:
Landing pad genotyping, for example by PCR amplification of the genomic locus of the host cell line;
Double payload transfection, for example by transfecting landing pad integrated cells with two payload plasmids, each encoding a different fluorescence protein;
Whole genome sequencing, for example by sequencing the entire genome of a candidate clone;
Multiplexed viral production, for example, by single-cell sorting a library of payload-integrated cells and infect cells with virus produced from each clone;
Control the multiplicity of infection (MOI) by for example, transducing cells at a low MOI, in some embodiments, an MOI of less than about 0.3;
Landing pad barcode amplification, by for example, sequencing the landing pad barcode of a candidate clone,
etc.

In some embodiments, a library of payload plasmids are transfected into the landing pad cell line comprising a plurality of landing pad cells (block 18). In some embodiments, the transfecting of the payload plasmids into the landing pad cell lines in block 18 comprises integrating the nucleic acid sequences of one payload plasmid into one of the landing pad cells.

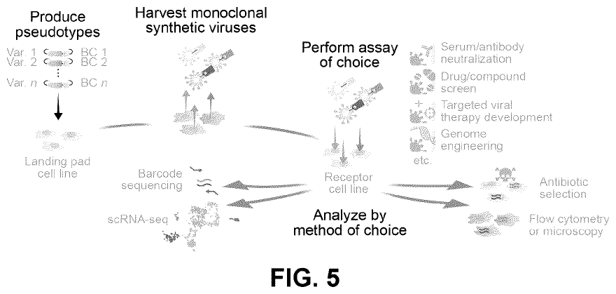

FIG. 5 is a schematic diagram illustrating non-limiting example downstream assays which may be performed on the library of functional viruses produced using the method and system disclosed herein. The downstream assay allows for high-throughput and large-scale screens of a big number of variants.

In one example, deep amplicon sequencing may be performed on the unique barcode sequences following transduction. In another example, scRNA-seq by hardcoding a 3′ polyadenylation tail downstream of the vBC (or unique barcode sequences) may be performed. Transcriptome-wide measurement of infected and uninfected cells may be performed for understanding the interplay between cell state and viral susceptibility. Further, in embodiments in which the functional viruses encode the one or more selectable markers (e.g., antibiotic resistance gene such as Puromycin) and/or reporter protein (e.g., fluorescence marker such as StayGold), one or more of antibiotic selection, flow cytometry, or fluorescence microscopy, etc. may be performed on the infected cells.

The invention is further described with reference to the following specific examples, which are not meant to limit the invention, but rather to further illustrate it.

### EXAMPLES

In these Examples, HEK293T cells were genetically modified by integration of a payload plasmid in a recombination site of a landing pad plasmid as schematically illustrated in FIG. 1D. In some examples, to promote integration of payloads, large serine integrases were used as the recombinase. Integrases are site-specific recombinases from bacteriophages that mediate the recombination reaction between two short (~40-100 bp) DNA sequences during the phage life cycle: the phage attachment site (attP) and bacterial attachment site (attB). After attB x attP recombination, the integrated prophage is flanked by residual DNA sequences attL and attR. Large serine integrases were used in the Examples to facilitate integration of large variant proteins, such as the SARS-COV-2 S protein, which is almost 4 kb long.

In some embodiments, a lentivirus vector system is used. In such embodiments, lentivirus is selected as the virus particle.

In some embodiments, a landing pad plasmid comprising a nucleic acid sequence that is set forth in SEQ ID NO. 1 was used. The landing pad plasmid was from pSK173.

In some embodiments, a VSV-G encoding payload plasmid comprising a nucleic acid sequence that is set forth in SEQ ID NO. 2 was used. The VSV-G encoding payload plasmid was from pSK176.

In some embodiments, the fully reconstituted GHOST LV genome comprises a nucleic acid sequence that is set forth in SEQ ID NO. 3. In such embodiments, the variant protein is the VSV-G protein.

In some embodiments, the method is optimized or adjusted to achieve integration of a single payload plasmid into each host cell. In some embodiments, the method is optimized or adjusted to allow sufficient expression of the viral components to only occur from the integrated payload. In some embodiments, the landing pad plasmid is designed as a dual promoter trap. The landing pad plasmid may comprise the common 5′ half of the LV genome with an attP site, and the payload plasmid may comprise the variable 3′ half of the transgenic LV genome with an attB site. Upon integration of the payload plasmid into the landing pad promoter trap, expression of the variant protein may be activated, and the full viral (e.g., LV) genome is reassembled (FIG. 1D). In some embodiments, the packaged virus (e.g., lentivirus) does not contain the variant protein in its genome, advantageously overcoming the bottleneck of packaging efficiency with increasing variant size.

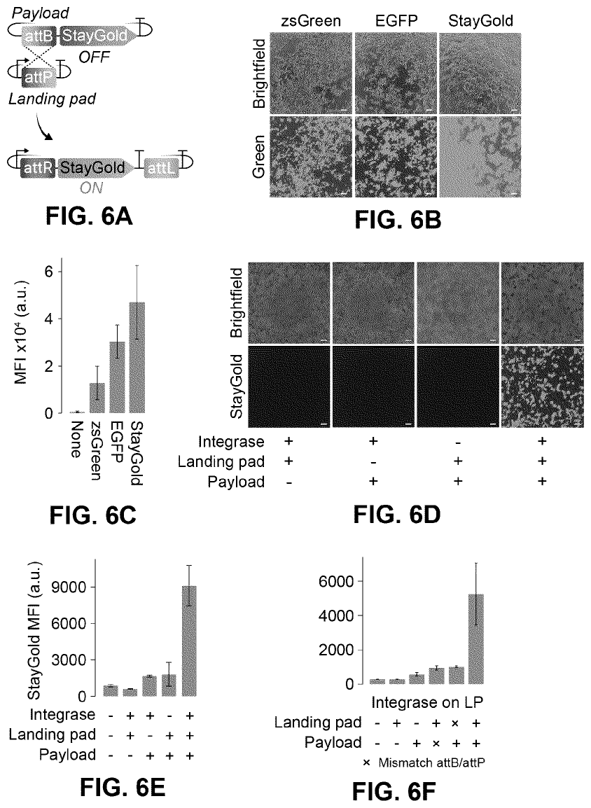

To test the method and systems of the present invention, a fluorescent payload integration reporter was established to enable measurement by flow cytometry or high-content imaging (FIGS. 6A-6F). The fluorophore StayGold was used (FIG. 6A-C). After transfecting the integrase Bxb1, as well as the payload and landing pad plasmids, StayGold median fluorescence intensity (MFI) was significantly higher in the presence of all three plasmids, indicating its utility as an integration reporter with minimal leak (FIG. 6D-E). When Bxb1 integrase was encoded on the landing pad plasmid, StayGold MFI was also the highest after transfection of the correct payload-landing pad pair (FIG. 6F). Additionally, a dual fluorescent reporter for the promoter trap using mTagBFP2 and StayGold was also created (FIG. 2B). A negative selection system that deactivates mCherry upon integration was also implemented FIG. 2A). StayGold expression was highly dependent on payload integration and mTagBFP2 MFI was significantly higher in the double transfection condition, suggesting that a pseudotype variant gene in place of mTagBFP2 would be strictly expressed upon integration (FIG. 2C-E). mCherry expression was reduced in the double transfection condition three days after transfection, benchmarking the negative selection system (FIG. 2D-E).

The reassembly of the split viral vectors was tested by performing a double transfection of payload, with or without VSV-G in place of mTagBFP2 (FIG. 2F), and landing pad plasmids in HEK293T cells. In some Examples, Bxb1 integrase was encoded on the landing pad plasmid to enhance transfection efficiency and later integration efficiency at the landing pad locus. Two days after the double transfection, the cells having the VSV-G-encoded payload were transfected with the LV packaging plasmid and the cells having no VSV-G with both the packaging and VSV-G pseudotyping plasmid. The viruses were harvested 2-3 days later, and were transduced into HEK293T cells. Cells having VSV-G supplemented exhibited a markedly stringent split virus reassembly and transduction compared to controls, including mismatched attB x attP sites and payloads missing a 3′LTR (FIG. 2G). Cells having the VSV-G-encoded payload also showed a significantly higher split virus reassembly but at a much lower efficiency, possibly due to the requirement for plasmid recombination before constitutive VSV-G expression, and because of the toxicity of VSV-G expressed in cells over the course of multiple days. These results indicate that even with many copies of landing pads and payloads in a single cell, as is the case with plasmid transfection, integration of the payload into the landing pad is specific enough and requires both 5′ and 3′ halves of the LV genome to reconstitute split synthetic viruses.

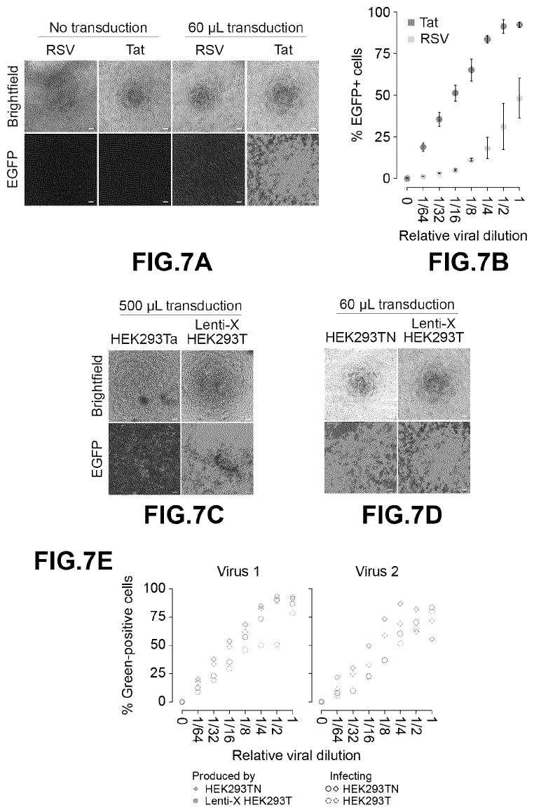

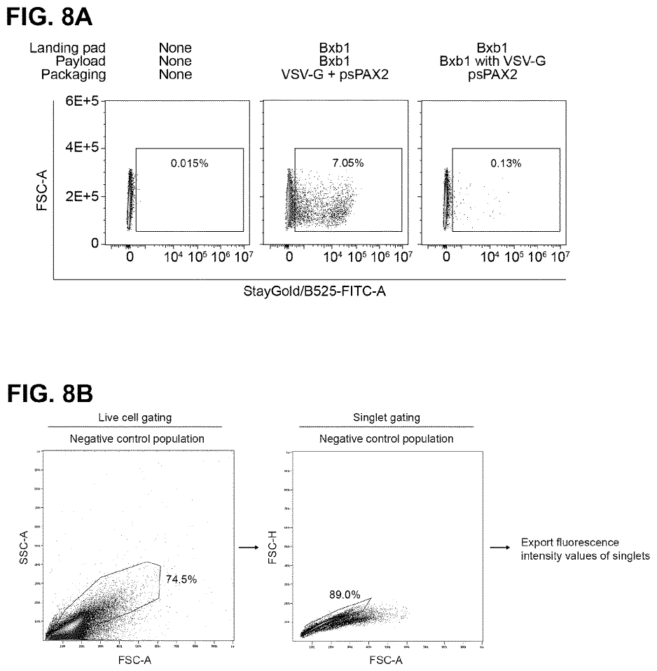

To showcase the modularity of GHOST, the integration efficiency of two serine recombinases, Bxb1 and Pa01, was compared. After transfecting HEK293T cells with the landing pad and payload, only cells transfected with matching attP and attB sites for Bxb1 and Pa01 showed StayGold expression (FIG. 2H). Pa01 showed an increase in StayGold mean fluorescence intensity across payload sizes of 4-10 kb (FIG. 2I). The promoters and cell lines used to express the LV genome and package the LV are also modular. For example, the RSV promoter can be replaced with a stronger Tat promoter for greater packaging efficiency (FIG. 7A-B), and the HEK293T cell line can be replaced with Lenti-X HEK293T or HEK293TN cells for improved lentiviral production efficiency (FIG. 7C-E).

Some Examples involve generating a landing pad cell line with a single recombination site for the payload plasmid. In some Examples, the landing pad cell line is generated by transcriptional activator-like effector nuclease (TALEN)-based knock-in at the AAVS1 safe harbor locus (see FIG. 3A). This method may be referred to hereinafter as “GHOST-SH”. Landing pad-transfected cells remaining after 13-days of Blasticidin treatment showed mCherry expression, indicating successful landing pad integration (FIG. 3B), and were expanded for two weeks before stocking and sorting for downstream assays (FIG. 3C). To validate virus production in these cells, the landing pad-transfected cells were transfected with a VSV-G encoding payload (FIG. 2F). 7 days post-transfection, the cells were selected by Puromycin for 6 days, further expanded, and assessed for payload integration by flow cytometry after pooling three biological replicates (FIG. 3D-E). Puromycin-selected cells were transfected with varying amounts of packaging plasmid (psPAX2) to assess their virus production capacity (FIG. 3F). Virus production showed a strict dependence on the packaging plasmid with 60-100% StayGold positive cells after VSV-G plasmid (pMD2.G) supplementation or 1-3% StayGold positive without (FIG. 3F).

To address the effects of using a heterogeneous cell population and to find a clone with single-copy landing pad integration, two single-cell sorts of the bulk Blasticidin-selected cells into 96-well plates were experimented (FIG. 3G). After successfully expanding 41 clones from the two sorts, analyses of mCherry expression by flow cytometry were performed to identify clones with unimodal mCherry expression profiles (FIG. 3H-I). Unimodal mCherry expression is preferable to ensure the variants of interest are as uniformly expressed as possible and suggests the cells are single-copy landing pad integrated. Twelve unimodal clones were assessed for successful landing pad integration by genotyping the AAVS1 locus (FIG. 3J). Primers were designed to flank the right and left homology arms for both the wildtype and landing pad-integrated AAVS1 loci (FIG. 3A). Candidate clones 24 (C24) and 35 (C35) showed the expected amplification at both the 5′ and 3′ landing pad integrated sites (FIG. 3J). After Sanger sequencing the ~1 kb band of the wildtype AAVS1 locus in C24 and C35, both clones revealed the same deletion that disrupted the left and right homology arm which could indicate non-homologous end joining of the AAVS1 locus upon TALEN-induced double stranded breaks (FIG. 3K-M).

To prepare candidate landing pad clones for virus production, the wildtype, unsorted landing pad, C24, and C35 cells were transfected with the VSV-G encoding payload (FIG. 2F). 6 days post-transfection, cells were selected by Puromycin treatment for 4 days, further expanded, and assessed for payload integration by flow cytometry (FIG. 3N). All of the cells without payload died and those with only payload were <1% StayGold positive. More heterogeneity was observed in the unsorted landing pad cells, which ranged from 14-96% Stay Gold positive, compared to 55-85% for C24, and 64-94% for C35 (FIG. 3N). Replicates with the highest % StayGold positive cells were stocked and used for subsequent virus production.

A virus production test was performed as before in FIGS. 3D-F except with candidate C35. Replicates 2, 1, and 3 of the wildtype, unsorted landing pad, and C35 cells, respectively, from FIG. 3N were used for virus production. Similarly to FIG. 3F, payload-transfected wildtype cells produced negligible virus whereas unsorted landing pad cells gave >3% StayGold positive cells (FIG. 3O). The lower StayGold positive cells from C35 may be a consequence of only having a single landing pad compared to the unsorted cells that may harbor multiple landing pad sites (FIG. 3O).

FIG. 4A is a schematic diagram illustrating a lentivirus integration strategy in the production of the landing pad cell line, referred to as “GHOST-LV”. FIG. 4B is an extension of GHOST-LV whereby Puromycin is reconstituted only after landing pad-payload recombination, ensuring strict selection of payload-integrated cells.

### Materials and Methods

### Plasmids

DNA synthesis and assembly. Oligonucleotides were chemically synthesized by Integrated DNA Technologies™. Large gene fragments were chemically synthesized by Twist Biosciences™. Plasmids were assembled by Gibson Assembly™ or Digestion-Ligation Assembly™. All plasmid DNA sequences were confirmed by Sanger sequencing by Azenta Life Sciences™ or Nanopore sequencing by Plasmidsaurus™

### Cell Culture

HEK293T cells. Human embryonic kidney (HEK) 293T Lenti-X cells, HEK293TN cells, and HEK293Ta cells were purchased from Takara™ (#632180), and GeneCopoeia™ (#LT008), respectively. Cells were cultured in Dulbecco's Modified Eagle Medium (DMEM) (Sigma-Aldrich™ #11965084) supplemented with 10% Fetal Bovine Serum (FBS) (Gibco™ #16000044) and 1% Penicillin-Streptomycin (P/S) (Gibco™ #15070063) at 37° C. with 5% CO₂ in a cell culture incubator. For passaging, cells were washed with 1× phosphate buffered saline (PBS) and detached by 0.25% w/v Trypsin-EDTA (Gibco™ #25200072) when the cells reached 70-90% confluency. For making cell stocks, cells were washed with 1×PBS, detached by 0.25% Trypsin-EDTA, resuspended in 5 mL of cell culture medium, and counted by TC20 Automated Cell Counter (Bio-Rad). Either ~1 or 2 million cells were aliquoted into 1.5-mL tubes and centrifuged at 1,000 rpm for 3 min. After aspirating the supernatant, the cells were resuspended in 1 mL of CELLBANKER 1™ (AMSBIO™ #11910), transferred into 1.2 mL cryogenic vials (Corning™ #430487), and stored at −80° C. or in a liquid nitrogen tank. Cell lines were regularly tested for mycoplasma contamination.

Genomic DNA preparation. About 1-2 million cells were aliquoted into 1.5-mL tubes and centrifuged at 1,000 rpm for 3 min. After aspirating the supernatant, the cells were washed with 1× phosphate buffered saline (PBS) and centrifuged again at 1,000 rpm. The supernatant was aspirated and the cell pellets were stored at −20° C. The genomic DNA was extracted using the Monarch Genomic DNA Purification Kit (New England Biolabs #T3010) according to the manufacturer's protocol.

### Transient Plasmid Delivery

Transfection of HEK293T cells. Cells were seeded ~18 h prior to transfection and were generally used only if healthy (>98% live) and at a low passage number of less than ~10. Transfection mixtures were prepared by mixing plasmid DNA with PEI MAX (Polysciences™ #24765-100) at a ratio of 3 μL of PEI MAX per 1 μg of DNA, dissolved in 1×PBS to a total volume of 500 μL (6-well plate), 50 μL (24-well plate), or 25 μL (96-well plate). The mixtures were then incubated at room temperature for 20 min, and added directly to the wells.

### Viral Production and Transduction

Lentiviral packaging and harvest. For general lentiviral production, 200,000 Lenti-X HEK293T cells were seeded in 2 mL into 6-well cell culture plates (Corning™ #3516). The following day, 366.7 ng of psPAX2 packaging plasmid (Addgene plasmid #12260), 122.3 ng of pMD2.G VSV-G glycoprotein plasmid (Addgene plasmid #12259), and 489 ng of lentiviral transfer plasmid were transfected together in 300 μL volumes. Approximately 18 h after transfection, the media was replaced with fresh cell culture media. The third morning after media exchange, the media was collected into 1.5-mL tubes and centrifuged at 1,000 rpm and room temperature for 5 min. The crude supernatants were aliquoted into 2 mL cryogenic vials (Corning™ #430488) and stored at −80° C. See each assay for their specific packaging and harvest procedures.

Viral titration. The day before transduction, 10,000 HEK293T cells were seeded in 50 μL volumes in 96-well flat-bottom cell culture plate wells (VIOLAMO #2018002). To determine viral titer, serial 1:2 viral dilutions in 60 UL volumes of cell culture media were transduced across the plate and transduction efficiencies based on fluorescence intensity were measured by flow cytometry. The viral titer was determined from the linear portion of the titration curve by multiplying the number of cells at the time of transduction with the number of cells infected and dividing the product by the volume (mL) of virus transduced.

Lentiviral transduction. The day before transduction, HEK293T cells were seeded according to the cell culture plate size used. Harvested viruses were added to a mixture of cell culture medium with 10 μg/mL Polybrene™ (Sigma-Aldrich™ #TR-1003-G) to a total volume equal to half of the cell culture medium volume present in the plate wells, and the viral mixture was directly added to the wells. See each assay for their specific transduction procedures.

### Microscopy

Sample preparation for microscopy. For nuclear counterstaining of HEK293T cells, 1× Hoechst 33342 Fluorescent Stain (Invitrogen™ #H3570) was dissolved in cell culture medium and directly added to the cells for 15 min. If no staining was performed, cells were washed with 1×PBS and fresh cell culture medium was added before imaging.

Live cell imaging. Brightfield and fluorescence microscopy of live cells was performed using BZ-X710 (Keyence™) with a 4× or 20× objective lens.

### Flow Cytometry

Sample preparation for flow cytometry. Cells were washed with 1×PBS, detached by 0.25% w/v Trypsin-EDTA (Gibco™ #2500072), incubated at 37° C. for 5 min., resuspended in cell culture medium and collected into 1.5-mL tubes or a 96-well V-bottom plate (Thermo Fisher Scientific™ #249570), and centrifuged at 1,000 rpm and room temperature for 5 min. After aspirating the supernatant, cell pellets were again gently resuspended with 200 μL ice-cold FACS buffer consisting of 2% FBS in 1×PBS. Samples were immediately placed on ice until flow cytometry analysis.

Flow cytometry analysis. Flow cytometry analysis was performed with BD FACSVerse Cell Analyzer™ (BD Biosciences™) or CytoFLEX Flow Cytometer™ (Beckman Coulter™). The approximate total volume was acquired for each sample. Data analysis was performed with FlowJo™ (version 10.8.2) or Cytobank™. A typical gating hierarchy was as follows: live cells were gated by FSC-A and SSC-A, singlets were gated by FSC-A and FSC-H, and fluorescent cells were gated for high fluorescence intensity when compared with an appropriate negative control sample with a lower expected fluorescence intensity.

### Landing Pad Cell Line Generation

Transfection of HEK293T cells. Low-passage Lenti-X HEK293T cells were seeded in 6-well cell culture plates at a density of ~200,000 cells/well in 2 mL cell culture medium. The following day, cells were transfected with 333 ng each of the left and right TALEN homology arm (HA) plasmids and 333 ng of landing pad plasmid, for a total of 999 ng of DNA. The HA plasmids target the AAVS1 safe harbor locus for DSB by TALEN.

Selection of landing pad cells. Five days after transfection, cells were washed twice with 1×PBS and 2 mL of cell culture medium with 5 μg/mL Blasticidin (Invivogen™ #ant-bl-05) was added to the cells. Every following day, the cells were again washed twice with 1×PBS and given fresh 5 μg/mL Blasticidin cell culture medium. On the sixth day of Blasticidin selection, the cells were passaged to a new 6-well cell culture plate. Each day after passaging, the cells were washed once with 1×PBS and given fresh 5 μg/mL Blasticidin cell culture medium. After 13 days of selection, Blasticidin-resistant colonies were apparent and expressed mCherry (FIG. 3B). After 14 days of expansion, the cells were stocked and sorted.

Flow cytometry cell sorting. Cells were washed twice with 1×PBS and detached by 0.25% w/v Trypsin-EDTA (Gibco™ #2500072), incubated at 37° C. for 5 min., and resuspended in 5 mL of cell culture medium. Cells were counted by TC20 Automated Cell Counter™ (Bio-Rad™) and approximately 500,000 cells were collected into a 1.5-mL tube and centrifuged at 1,000 rpm and room temperature for 5 min. After aspirating the supernatant, the cells were resuspended into a 5-mL polystyrene round-bottom tube (Falcon™ #14-959-1A) in 500 μL of ice-cold FACS buffer consisting of 2% FBS in 1×PBS. The cell suspension was immediately placed on ice until sorting. Cell sorting was performed using BD FACSJazz (BD Biosciences) with 1.0 Drop Single Sort mode. Live cells were first gated by FSC-A and SSC-A, and then single cells by FSC-A and FSC-H. Cells were gated for high mCherry intensity when compared with a negative control sample with a lower expected fluorescence intensity. Single mCherry positive cells were sorted into all wells of a 96-well flat-bottom cell culture plate (VIOLAMO #2018002), each containing 2.5 μL of 1×PBS. The samples were immediately placed on ice and 200 μL of cell culture medium was added to each well after sorting was complete. Cells were kept at 37° C. with 5% CO₂ in a cell culture incubator and expanded for about 14 days before making cell stock.

Landing pad genotyping. PCRs were performed with 10 μL HiFi HotStart ReadyMix (KAPA BIOSYSTEMS #KK2601), 0.6 UM each primer, and 25-100 ng genomic DNA to a total of 20 μL reaction volumes. Reaction conditions were 95° C. 3′, 98° C. 20″, 60-72° C. 15″, 72° C. 15″-30″, steps 2-4 repeated 34 times, 72° C. 5′, 4° C. hold. Reactions were run on 1-2% TBE/agarose gels and visualized using SYBR Safe (ThermoFisher Scientific #S33102). Gel bands were purified using the GeneJET Gel Purification Kit (ThermoFisher Scientific #K0702). The primer sequences used for landing pad genotyping are listed in the table below.

TABLE 1
Landing Pad Genotyping Primer Sequences

| Primer | Sequence | SEQ ID NO. |
|---|---|---|
| a | cgctgccgtctctctcctgagtccgg | SEQ ID NO. 4 |
| b | gcagcagtgcccagcaccacgagttctg | SEQ ID NO. 5 |
| C | ctctcactcggaaggacatatgggagggca | SEQ ID NO. 6 |
| d | ggccacgtaacctgagaagggaatccc | SEQ ID NO. 7 |
| b′ | acccagaaccagagccacattaaccggccc | SEQ ID NO. 8 |
| c′ | ggacaggattggtgacagaaaagccccatc | SEQ ID NO. 9 |
| β-actin FW | cctgggcaggtcggctgtggggtcc | SEQ ID NO. 10 |
| β-actin RV | gcgaggccaggatggagccgccg | SEQ ID NO. 11 |
| Landing pad plasmid FW | tgcctgcttgccgaatatca | SEQ ID NO. 12 |
| Landing pad plasmid RV | taccgcacagatgcgtaagg | SEQ ID NO. 13 |

### Payload-Landing Pad Plasmid Recombination Assays

Fluorescent reporter integration. Approximately 20,000 Lenti-X HEK293T cells were seeded in 200 UL volumes in 96-well flat-bottom cell culture plate wells (VIOLAMO™ #2018002). For recombination of payloads and landing pads with no integrase, 200 ng of integrase, 70 ng of landing pad, and 50 ng of payload were transfected together in 25 μL volumes the following day. For recombination of payloads and landings with integrase, 200 ng of landing pad and 50 ng of payload were transfected together in 25 μL volumes the following day. The cells were analyzed by flow cytometry about 3 days after transfection.

Split viral vector packaging and transduction. Approximately 650,000 Lenti-X HEK293T cells were seeded into 6-well cell culture plate wells (Corning™ #3516) in 2 mL volumes. The following day, 500 ng of payload and 500 ng of landing pad were transfected together in 500 μL volumes. Two days after the first transfection, the media was replaced with fresh cell culture media and 500 ng of psPAX2 was transfected in 500 μL volumes (if payload plasmids encoded VSV-G), or 500 ng of psPAX2 and 500 ng of pMD2.G were transfected together in 500 μL volumes (if payload plasmids did not encode VSV-G). Three days after the second transfection, the cell culture media was collected and concentrated using Lenti-X Concentrator (Takara™ #631232) to 30 μL volumes following the manufacturer's protocol and split equally into three replicates. The day before transduction, approximately 50,000 Lenti-X HEK293T cells were seeded into 24-well cell culture plate wells (Corning™ #3524) in 1 mL volumes. The concentrated viruses were added to a mixture of cell culture medium with 10 μg/mL Polybrene (Sigma-Aldrich™ #TR-1003-G) to a total volume of ~500 UL and the viral mixture was directly added to the wells. Two days after transduction, the cells were analyzed by flow cytometry.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms. These terms (“a”, “an”, and “the”) mean one or more unless stated otherwise;
“and/or” is used to indicate one or both stated cases may occur, for example A and/or B includes both (A and B) and (A or B);
“approximately” when applied to a numerical value means the numerical value ±10%;
where a feature is described as being “optional” or “optionally” present or described as being present “in some embodiments” it is intended that the present disclosure encompasses embodiments where that feature is present and other embodiments where that feature is not necessarily present and other embodiments where that feature is excluded. Further, where any combination of features is described in this application this statement is intended to serve as antecedent basis for the use of exclusive terminology such as “solely,” “only” and the like in relation to the combination of features as well as the use of “negative” limitation(s)” to exclude the presence of other features; and
“first” and “second” are used for descriptive purposes and cannot be understood as indicating or implying relative importance or indicating the number of indicated technical features.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a range for a value is stated, the stated range includes all sub-ranges of the range. It is intended that the statement of a range supports the value being at an endpoint of the range as well as at any intervening value to the tenth of the unit of the lower limit of the range, as well as any subrange or sets of sub ranges of the range unless the context clearly dictates otherwise or any portion(s) of the stated range is specifically excluded. Where the stated range includes one or both endpoints of the range, ranges excluding either or both of those included endpoints are also included in the invention.

Certain numerical values described herein are preceded by “about”. In this context, “about” provides literal support for the exact numerical value that it precedes, the exact numerical value ±5%, as well as all other numerical values that are near to or approximately equal to that numerical value. Unless otherwise indicated a particular numerical value is included in “about” a specifically recited numerical value where the particular numerical value provides the substantial equivalent of the specifically recited numerical value in the context in which the specifically recited numerical value is presented. For example, a statement that something has the numerical value of “about 10” is to be interpreted as: the set of statements:
in some embodiments the numerical value is 10;
in some embodiments the numerical value is in the range of 9.5 to 10.5;
   and if from the context the person of ordinary skill in the art would understand that values within a certain range are substantially equivalent to 10 because the values with the range would be understood to provide substantially the same result as the value 10 then “about 10” also includes:
in some embodiments the numerical value is in the range of C to D where C and D are respectively lower and upper endpoints of the range that encompasses all of those values that provide a substantial equivalent to the value 10.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

As will be apparent to those of skill in the art upon reading this disclosure, each of the individual embodiments described and illustrated herein has discrete components and features which may be readily separated from or combined with the features of any other described embodiment(s) without departing from the scope of the present invention.

Any aspects described above in reference to apparatus may also apply to methods and vice versa.

Any recited method can be carried out in the order of events recited or in any other order which is logically possible. For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, simultaneously or at different times.

Various features are described herein as being present in “some embodiments”. Such features are not mandatory and may not be present in all embodiments. Embodiments of the invention may include zero, any one or any combination of two or more of such features. All possible combinations of such features are contemplated by this disclosure even where such features are shown in different drawings and/or described in different sections or paragraphs. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible). This is the case even if features A and B are illustrated in different drawings and/or mentioned in different paragraphs, sections or sentences.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. A method for generating a synthetic virus, comprising the steps of:
(a) producing a landing pad cell line engineered from a host cell line, wherein the landing pad cell comprises a nucleic acid sequence having a first segment of a genome of a virus particle, and a first recombination site, wherein the first segment of the genome of the virus particle and the first recombination site are inserted into a genomic locus of the host cell; and
(b) transfecting a payload plasmid into the landing pad cell to produce a payload- and landing pad-integrated cell, wherein the payload plasmid comprises a nucleic acid sequence having a second segment of the genome of the virus particle, a second recombination site, and a nucleic acid sequence which encodes a variant protein, wherein the transfecting of the landing pad cell with the payload plasmid comprises recombining the nucleic acid sequences contained in the payload plasmid and the landing pad cell in the presence of a site-specific recombinase,
wherein the payload- and landing pad-integrated cell comprises a nucleic acid sequence encoding a genome of a reassembled virus particle comprising the recombined first and second segments of the genome of the virus particle, and wherein the expression of the variant protein is activated upon payload plasmid recombination within the landing pad cell.

2. The method according to claim 1, wherein the variant protein is encoded outside of the genome of the reassembled virus particle.

3. The method according to claim 1, further comprising (c) harvesting a virus from the payload- and landing pad-integrated cell; and (d) transducing the virus into a target cell line.

4. (canceled)

5. The method according to claim 1, further comprising:
(e) transfecting a packaging plasmid into the payload- and landing pad-integrated cell to produce a transfected cell; and
(f) harvesting a functional virus from the transfected cell.

6. The method according to claim 1,
wherein the nucleic acid sequence of the payload plasmid further comprises a unique barcode sequence, and wherein the unique barcode sequence is encoded in the genome of the reassembled virus particle.

7. (canceled)

8. The method according to claim 1,
wherein the landing pad cells and/or the payload plasmids further comprises nucleic acid sequences encoding one or more selectable markers and/or reporter proteins, and wherein the method further comprises (g) selecting, after the transfecting step (b), for the payload- and landing pad-integrated cells expressing the selectable marker and/or reporting protein, and transfecting in step (e), the packaging plasmids into the selected payload- and landing pad-integrated cells.

9.-13. (canceled)

14. The method according to claim 5, further comprising (i) transducing the functional virus into a target cell line.

15. (canceled)

16. The method according to claim 1,
wherein the first segment of the genome of the virus particle comprises the 5′ LTR region, and one or more of a Psi packaging element (Psi), Rev responsive element (RRE), and Tat responsive element (TAR).

17. The method according to claim 16,
wherein the first segment of the genome of the virus particle further comprises a lentiviral central polypurine tract (cPPT).

18.-19. (canceled)

20. The method according to claim 1,
wherein a constitutive promoter is operably linked to the first recombination site.

21. The method according to claim 1,
wherein two constitutive promoters are operably linked to the first recombination site, wherein a first constitutive promoter is operably linked upstream of the first recombination site in a first direction, and a second constitutive promoter is operably linked downstream of the first recombination site in an opposing second direction.

22.-24. (canceled)

25. The method according to claim 1,
wherein the second segment of the genome of the virus particle comprises the 3′ LTR region, and a posttranscriptional regulatory element sequence.

26. (canceled)

27. The method according to claim 1,
wherein the landing pad cell further comprises a nucleic acid sequence which encodes a recombinase.

28.-31. (canceled)

32. The method according to claim 5,
wherein the transfecting of the payload plasmid into the landing pad cell in step (e) comprises recombining the nucleic acid sequences contained in the landing pad cell and the payload plasmid at the first and second recombination sites to form first and second recombined sites.

33. (canceled)

34. The method according to claim 32, wherein the nucleic acid sequence which encodes the variant protein is integrated between the 3′ LTR region of the genome of the reassembled virus particle and the first recombined site.

35. (canceled)

36. The method according to claim 6,
wherein the barcode sequence is integrated between the 3′ LTR region of the genome of the reassembled virus particle and the second recombined site.

37.-39. (canceled)

40. The method according to claim 36,
wherein the unique barcode sequence comprises a DNA sequence with the formula, (SW)ₙ, wherein S is guanine (G) or cytosine (C), and W is adenine (A) thymine (T), and n is at least 1.

41. (canceled)

42. The method according to claim 1,
wherein a constitutive promoter or an inducible promoter is operably linked to the nucleic acid encoding the variant protein.

43.-44. (canceled)

45. The method according to claim 1,
wherein the genomic locus of the host cell is a safe harbor locus, wherein the safe harbor locus is selected from one or more of AAVS1, CCR5, ROSA26, collagen, HTRP, H11, beta-2 microglobulin, GAPDH, TCR, RUNX1, and ALB.

46. (canceled)

47. The method according to claim 1,
wherein the genomic locus of the host cell is in a transcriptional unit.

48.-141. (canceled)


SEQUENCE LISTING (13 sequences)

SEQ ID NO: 1 - DNA, 9390 bp, synthetic construct
  source 1..9390: mol_type = other DNA; note = HA-L + Blasticidin resistance gene + 5 prime LV genome (Tat responsive element + 5 prime LTR + Psi element + Rev responsive element + cPPT) + EF1-alpha promoter + Bxb1 attP site + CMV promoter + Bxb1 integrase + P2A + mCherry + HA-R; note = Landing pad (from pSK173)
gcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccaagcttctgacctcttctcttcctcccacagggcctcgagagatctggcagcggagagggcagaggaagtcttctaacatgcggtgacgtggaggagaatcccggccctaggctcgagatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaatctagacagggttattgtctcatgagcggatacatatttgaatgtatttagaaaaataaacaaataggggttccgcgcacatttccccgaaaagtgccacctgacgtcgacggatcgggagatcaactttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattagccgggtttgtctggtcaaccaccgcggtctcagtggtgtacggtacaaaccgccaccatggcggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccggcgcgccatgccaaaaaagaaaagaaaagtgtatccctatgatgtccccgattatgccggttcaagagccctggtcgtgattagactgagccgagtgacagacgccaccacaagtcccgagagacagctggaatcatgccagcagctctgtgctcagcggggttgggatgtggtcggcgtggcagaggatctggacgtgagcggggccgtcgatccattcgacagaaagaggaggcccaacctggcaagatggctcgctttcgaggaacagccctttgatgtgatcgtcgcctacagagtggaccggctgacccgctcaattcgacatctccagcagctggtgcattgggctgaggaccacaagaaactggtggtcagcgcaacagaagcccacttcgatactaccacaccttttgccgctgtggtcatcgcactgatgggcactgtggcccagatggagctcgaagctatcaaggagcgaaacaggagcgcagcccatttcaatattagggccggtaaatacagaggctccctgcccccttggggatatctccctaccagggtggatggggagtggagactggtgccagaccccgtccagagagagcggattctggaagtgtaccacagagtggtcgataaccacgaaccactccatctggtggcacacgacctgaatagacgcggcgtgctctctccaaaggattattttgctcagctgcagggaagagagccacagggaagagaatggagtgctactgcactgaagagatctatgatcagtgaggctatgctgggttacgcaacactcaatggcaaaactgtccgggacgatgacggagcccctctggtgagggctgagcctattctcaccagagagcagctcgaagctctgcgggcagaactggtcaagactagtcgcgccaaacctgccgtgagcaccccaagcctgctcctgagggtgctgttctgcgccgtctgtggagagccagcatacaagtttgccggcggagggcgcaaacatccccgctatcgatgcaggagcatggggttccctaagcactgtggaaacgggacagtggccatggctgagtgggacgccttttgcgaggaacaggtgctggatctcctgggtgacgctgagcggctggaaaaagtgtgggtggcaggatctgactccgctgtggagctggcagaagtcaatgccgagctcgtggatctgacttccctcatcggatctcctgcatatagagctgggtccccacagagagaagctctggacgcacgaattgctgcactcgctgctagacaggaggaactggagggcctggaggccaggccctctggatgggagtggcgagaaaccggacagaggtttggggattggtggagggagcaggacaccgcagccaagaacacatggctgagatccatgaatgtccggctcacattcgacgtgcgcggtggcctgactcgaaccatcgattttggcgacctgcaggagtatgaacagcacctgagactggggtccgtggtcgaaagactgcacactgggatgtccggtaccggcagcggcgccaccaacttcagcctgctgaagcaggccggcgacgtggaggagaaccccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgcgttgaacttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 2 - DNA, 7453 bp, synthetic construct
  source 1..7453: mol_type = other DNA; note = VSV-G encoding payload (from pSK176); note = VSV-G + Bxb1 attB site + Puromycin resistance gene + P2A + StayGold + Variant barcode + PolyA + 3 prime LV genome (WPRE + 3 prime LTR) + Payload plasmid backbone
ttactttccaagtcggttcatctctatgtctgtataaatctgtcttttcttggtgtgctttaatttaatgcaaagatggataccaactcggagaaccaagaatagtccaatgattaaccctatgataaagaaaaaagaggcaatagagcttttccaactactgaaccaaccttctacaagctcgattggatttttggatagcccagtatcaccaaaaaataaactctcatcatcaggaagttgcgaagcagcgtcttgaatgtgaggatgttcgaacacctgagcctttgagctaagatgaagatcggagtccaacataccatgtccaatcatgtataaaggaaacttatatcctgaactggtcctcagaactccattgggtccaatttccacgtcttcatatggtgcccagtcatcccacagttccctttctgtggtagttccactgatcattccgaccattcttgagaggattggagcagcaatatcgactctgatgtatctggtctcaaagtattttagggtaccattgattatggtgaaagcaggaccggttcctgggtttttaggagcaagatagctgagatccactggagagattggaagacccgctctgattttgctccaggtttcttggcagagggaataatccaagatcctctcaacgtcctgaattagacttacatccactgaggtctgagatggagcagagatacttgacccttctgggcattcagggaatctggctgcagcaaagagatccttatcagccatctcgaaccagacacctgatgggagtctgactccccaatgcttgcagtattgcattttgcaggccttgcctccagtttcataagcaaagtagttacttctgaaccctgtgccctcctttcccagggatgatagctctccgtcctctgagaagaaggtgatgtccatggaaatgaggttagaatcacatagccctttgaccttatagtcagaatgccaggttgtagagttatggacagtggggcatatgtaattgctgcattttccgttgatgaactgtgaatcaacccattctcctgtgtattcatcaaccagcacatggtgaggagtcacctggacaatcactgcttcggcatccgtcacagttgcatatccacaactttgaggagggaagcctggattcagccaagttccttgtttcgtttgttcaatgctttccttgcattgttctacagatggagtgaaggatcggatggaatgtgttatatacttcggtccataccagcggaaatcacaagtagtgacccatttggaagcatgacacatccaaccgtctgcttgaatagccttgtgactcttgggcattttgacttgtaaggctgtgcctattaagtcattatgccaatttaaatctgagcttgacgggcaataatggtaattagaaggaacatttttccagtttcctttttggttgtgtggaaaaactatggtgaacttgcaattcaccccaatgaataaaaaggctaagtacaaaaggcacttcatggcgcgcctgatcagccgatcgcattacgccaagcgcgcaattaaccctcactaaagggcctgcagggccggcttgtcgacgacggcggtctccgtcgtcaggatcatccgcctaggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgaattcggcagcggcgccaccaacttcagcctgctgaagcaggccggcgacgtggaggagaaccccggccccctcgagatggccagcacccccttcaagttccagctgaagggcaccatcaacggcaagagcttcaccgtggaaggcgagggcgagggcaatagccacgagggcagccacaaaggcaagtacgtgtgcaccagcggcaaactgccaatgtcttgggccgccctgggaactagcttcggctatggcatgaagtactacaccaagtaccccagcggcctgaagaactggttccacgaggtgatgcccgagggcttcacctacgacagacacatccagtacaagggcgacggcagcatccacgccaagcaccagcacttcatgaagaacggcacctaccacaacatcgtggagttcaccggccaggacttcaaggagaacagccccgtgctgaccggcgacatgaacgtgagcctgcccaacgaggtgcagcacatccccagagatgacggcgtggagtgcccagtgaccctgctgtaccctctgctgagcgacaagagcaagtgcgtggaggcccaccagaacaccatctgcaagcccctgcacaatcagccagcccccgatgtgccataccactggatcagaaagcagtacacccagagcaaggacgacaccgaggagagagaccacatctgccagagcgagaccctggaggcccacctgtaataactataacggtcctaaggtagcgaatctagataacttacggagtcgctctacggtcagtgtgagtgtgtcagtgtgagtgtcagtgtctcacagtgtcagagagacaggacctaaagaatcccatcccaagtgttcggatccattaccctgttatccctactcgaactcgcatacctctgataacaaaaaaaaaaaaaaaaaaaaaaaaaaaaaaaaaaaaaaaaaaaaaaaaaaaaaaaaaaaagatccaattctaccgggtaggggaggcgcttttcccaaggcagtctggagcatgcgctttagcagccccgctgggcacttggcgctacacaagtggcctctggcctcgcacacattccacatccaccggtagacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgttttaccgtcgacctctagctagagcttggcgtaatgcgatcgcggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagtattcaaataccgtattgaacgcggccgcccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaaattaatacgactcactatagggagacccaccggtg

SEQ ID NO: 3 - DNA, 16843 bp, synthetic construct
  source 1..16843: mol_type = other DNA; note = Fully reconstituted GHOST LV genome; note = HA-L + Blasticidin resistance gene + 5 prime LV genome (Tat responsive element + 5 prime LTR + Psi element + Rev responsive element + cPPT) + EF1-alpha promoter + Bxb1 attR site + Puromycin resistance gene + P2A + StayGold + Variant barcode + PolyA + 3 prime LV genome (WPRE + 3 prime LTR) + Payload plasmid backbone + VSV-G + Bxb1 attL site + CMV promoter + Bxb1 integrase + P2A + mCherry + HA-R
gcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccaagcttctgacctcttctcttcctcccacagggcctcgagagatctggcagcggagagggcagaggaagtcttctaacatgcggtgacgtggaggagaatcccggccctaggctcgagatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaatctagacagggttattgtctcatgagcggatacatatttgaatgtatttagaaaaataaacaaataggggttccgcgcacatttccccgaaaagtgccacctgacgtcgacggatcgggagatcaactttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattagccgggtttgtctggtcaaccaccgcggtctccgtcgtcaggatcatccgcctaggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgaattcggcagcggcgccaccaacttcagcctgctgaagcaggccggcgacgtggaggagaaccccggccccctcgagatggccagcacccccttcaagttccagctgaagggcaccatcaacggcaagagcttcaccgtggaaggcgagggcgagggcaatagccacgagggcagccacaaaggcaagtacgtgtgcaccagcggcaaactgccaatgtcttgggccgccctgggaactagcttcggctatggcatgaagtactacaccaagtaccccagcggcctgaagaactggttccacgaggtgatgcccgagggcttcacctacgacagacacatccagtacaagggcgacggcagcatccacgccaagcaccagcacttcatgaagaacggcacctaccacaacatcgtggagttcaccggccaggacttcaaggagaacagccccgtgctgaccggcgacatgaacgtgagcctgcccaacgaggtgcagcacatccccagagatgacggcgtggagtgcccagtgaccctgctgtaccctctgctgagcgacaagagcaagtgcgtggaggcccaccagaacaccatctgcaagcccctgcacaatcagccagcccccgatgtgccataccactggatcagaaagcagtacacccagagcaaggacgacaccgaggagagagaccacatctgccagagcgagaccctggaggcccacctgtaataactataacggtcctaaggtagcgaatctagataacttacggagtcgctctacggtcagtgtgagtgtgtcagtgtgagtgtcagtgtctcacagtgtcagagagacaggacctaaagaatcccatcccaagtgttcggatccattaccctgttatccctactcgaactcgcatacctctgataacaaaaaaaaaaaaaaaaaaaaaaaaaaaaaaaaaaaaaaaaaaaaaaaaaaaaaaaaaaaagatccaattctaccgggtaggggaggcgcttttcccaaggcagtctggagcatgcgctttagcagccccgctgggcacttggcgctacacaagtggcctctggcctcgcacacattccacatccaccggtagacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgttttaccgtcgacctctagctagagcttggcgtaatgcgatcgcggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagtattcaaataccgtattgaacgcggccgcccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaaattaatacgactcactatagggagacccaccggtgttactttccaagtcggttcatctctatgtctgtataaatctgtcttttcttggtgtgctttaatttaatgcaaagatggataccaactcggagaaccaagaatagtccaatgattaaccctatgataaagaaaaaagaggcaatagagcttttccaactactgaaccaaccttctacaagctcgattggatttttggatagcccagtatcaccaaaaaataaactctcatcatcaggaagttgcgaagcagcgtcttgaatgtgaggatgttcgaacacctgagcctttgagctaagatgaagatcggagtccaacataccatgtccaatcatgtataaaggaaacttatatcctgaactggtcctcagaactccattgggtccaatttccacgtcttcatatggtgcccagtcatcccacagttccctttctgtggtagttccactgatcattccgaccattcttgagaggattggagcagcaatatcgactctgatgtatctggtctcaaagtattttagggtaccattgattatggtgaaagcaggaccggttcctgggtttttaggagcaagatagctgagatccactggagagattggaagacccgctctgattttgctccaggtttcttggcagagggaataatccaagatcctctcaacgtcctgaattagacttacatccactgaggtctgagatggagcagagatacttgacccttctgggcattcagggaatctggctgcagcaaagagatccttatcagccatctcgaaccagacacctgatgggagtctgactccccaatgcttgcagtattgcattttgcaggccttgcctccagtttcataagcaaagtagttacttctgaaccctgtgccctcctttcccagggatgatagctctccgtcctctgagaagaaggtgatgtccatggaaatgaggttagaatcacatagccctttgaccttatagtcagaatgccaggttgtagagttatggacagtggggcatatgtaattgctgcattttccgttgatgaactgtgaatcaacccattctcctgtgtattcatcaaccagcacatggtgaggagtcacctggacaatcactgcttcggcatccgtcacagttgcatatccacaactttgaggagggaagcctggattcagccaagttccttgtttcgtttgttcaatgctttccttgcattgttctacagatggagtgaaggatcggatggaatgtgttatatacttcggtccataccagcggaaatcacaagtagtgacccatttggaagcatgacacatccaaccgtctgcttgaatagccttgtgactcttgggcattttgacttgtaaggctgtgcctattaagtcattatgccaatttaaatctgagcttgacgggcaataatggtaattagaaggaacatttttccagtttcctttttggttgtgtggaaaaactatggtgaacttgcaattcaccccaatgaataaaaaggctaagtacaaaaggcacttcatggcgcgcctgatcagccgatcgcattacgccaagcgcgcaattaaccctcactaaagggcctgcagggccggcttgtcgacgacggcggtctcagtggtgtacggtacaaaccgccaccatggcggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccggcgcgccatgccaaaaaagaaaagaaaagtgtatccctatgatgtccccgattatgccggttcaagagccctggtcgtgattagactgagccgagtgacagacgccaccacaagtcccgagagacagctggaatcatgccagcagctctgtgctcagcggggttgggatgtggtcggcgtggcagaggatctggacgtgagcggggccgtcgatccattcgacagaaagaggaggcccaacctggcaagatggctcgctttcgaggaacagccctttgatgtgatcgtcgcctacagagtggaccggctgacccgctcaattcgacatctccagcagctggtgcattgggctgaggaccacaagaaactggtggtcagcgcaacagaagcccacttcgatactaccacaccttttgccgctgtggtcatcgcactgatgggcactgtggcccagatggagctcgaagctatcaaggagcgaaacaggagcgcagcccatttcaatattagggccggtaaatacagaggctccctgcccccttggggatatctccctaccagggtggatggggagtggagactggtgccagaccccgtccagagagagcggattctggaagtgtaccacagagtggtcgataaccacgaaccactccatctggtggcacacgacctgaatagacgcggcgtgctctctccaaaggattattttgctcagctgcagggaagagagccacagggaagagaatggagtgctactgcactgaagagatctatgatcagtgaggctatgctgggttacgcaacactcaatggcaaaactgtccgggacgatgacggagcccctctggtgagggctgagcctattctcaccagagagcagctcgaagctctgcgggcagaactggtcaagactagtcgcgccaaacctgccgtgagcaccccaagcctgctcctgagggtgctgttctgcgccgtctgtggagagccagcatacaagtttgccggcggagggcgcaaacatccccgctatcgatgcaggagcatggggttccctaagcactgtggaaacgggacagtggccatggctgagtgggacgccttttgcgaggaacaggtgctggatctcctgggtgacgctgagcggctggaaaaagtgtgggtggcaggatctgactccgctgtggagctggcagaagtcaatgccgagctcgtggatctgacttccctcatcggatctcctgcatatagagctgggtccccacagagagaagctctggacgcacgaattgctgcactcgctgctagacaggaggaactggagggcctggaggccaggccctctggatgggagtggcgagaaaccggacagaggtttggggattggtggagggagcaggacaccgcagccaagaacacatggctgagatccatgaatgtccggctcacattcgacgtgcgcggtggcctgactcgaaccatcgattttggcgacctgcaggagtatgaacagcacctgagactggggtccgtggtcgaaagactgcacactgggatgtccggtaccggcagcggcgccaccaacttcagcctgctgaagcaggccggcgacgtggaggagaaccccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgcgttgaacttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 4 - DNA, 26 bp, synthetic construct
  source 1..26: mol_type = other DNA; note = Primer a
cgctgccgtctctctcctgagtccgg

SEQ ID NO: 5 - DNA, 28 bp, synthetic construct
  source 1..28: mol_type = other DNA; note = Primer b
gcagcagtgcccagcaccacgagttctg

SEQ ID NO: 6 - DNA, 30 bp, synthetic construct
  source 1..30: mol_type = other DNA; note = Primer c
ctctcactcggaaggacatatgggagggca

SEQ ID NO: 7 - DNA, 27 bp, synthetic construct
  source 1..27: mol_type = other DNA; note = Primer d
ggccacgtaacctgagaagggaatccc

SEQ ID NO: 8 - DNA, 30 bp, synthetic construct
  source 1..30: mol_type = other DNA; note = Primer b prime
acccagaaccagagccacattaaccggccc

SEQ ID NO: 9 - DNA, 30 bp, synthetic construct
  source 1..30: mol_type = other DNA; note = Primer c prime
ggacaggattggtgacagaaaagccccatc

SEQ ID NO: 10 - DNA, 25 bp, synthetic construct
  source 1..25: mol_type = other DNA; note = Primer Beta-actin FW
cctgggcaggtcggctgtggggtcc

SEQ ID NO: 11 - DNA, 23 bp, synthetic construct
  source 1..23: mol_type = other DNA; note = Primer Beta-actin RV
gcgaggccaggatggagccgccg

SEQ ID NO: 12 - DNA, 20 bp, synthetic construct
  source 1..20: mol_type = other DNA; note = Primer Landing pad plasmid FW
tgcctgcttgccgaatatca

SEQ ID NO: 13 - DNA, 20 bp, synthetic construct
  source 1..20: mol_type = other DNA; note = Primer Landing pad plasmid RV
taccgcacagatgcgtaagg